(19) United States
(12) Patent Application Publication
(10) Pub. No.: US 2008/0234659 A1
(43) Pub. Date: Sep. 25, 2008

(54) UROLOGICAL MEDICAL DEVICES FOR RELEASE OF THERAPEUTIC AGENTS

(75) Inventors: Eric Cheng, Bloomington, IN (US); Jianmin Li, Lexington, MA (US); Weena Bucay-Couto, Burlington, MA (US); Scott Sanders, Hinsdale, IL (US); James F. Schuermann, Natick, MA (US); Min-Shyan Sheu, Chelmsford, MA (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 12/052,037

(22) Filed: Mar. 20, 2008

Related U.S. Application Data
(60) Provisional application No. 60/919,081, filed on Mar. 20, 2007.

(51) Int. Cl. A61M 25/00; A61F 2/04

(52) U.S. Cl. 604/523; 623/23.66

(57) ABSTRACT

According to an aspect of the present invention, urological medical devices are provided, which contain one or more urologically beneficial agents selected from alpha-adrenergic blockers, calcium channel blockers, and combinations thereof, among others. The urological devices are adapted for implantation or insertion into a subject's urinary tract, whereupon at least a portion of the urologically beneficial agent is released. Such agents are urologically beneficial, for example, in that they may relieve pain and/or discomfort associated with the medical device and/or act as stone expulsion agents (i.e., they facilitate stone passage), among other benefits. According to an aspect of the present invention, a method of treating kidney stones is provided which comprises: (a) diagnosing the presence of kidney stones within a subject and (b) implanting or inserting a urological medical device into the subject which contains at least one urologically beneficial agent. The medical device is adapted to release the at least one urologically beneficial agent in vivo in an amount effective to promote kidney stone expulsion.

### RELATED APPLICATION SECTION

This application claims the benefit of U.S. Provisional Application Ser. No. 60/919,081, filed Mar. 20, 2007, entitled “Urological Medical Devices for Release of Therapeutic Agents,” which is incorporated by reference herein in its entirety.

### FIELD OF THE INVENTION

The present invention relates generally to urological medical devices, and more particularly to implantable or insertable urological medical devices which release therapeutic agents.

### BACKGROUND OF THE INVENTION

Various urological medical devices have been developed for implantation or insertion into patients. Such medical devices are commonly associated with some degree of patient discomfort or pain after being positioned within the patient. As a specific example, ureteral stents are widely used to facilitate drainage in the upper urinary tract (e.g., from the kidney to the bladder), for example, following ureteroscopy, endourerotomies, and endopyelotomy for ureteral strictures, as well as in other instances where ureteral obstruction may occur for example, following lithotripsy. Such stents, however, are commonly associated with pain and discomfort in the bladder and flank area after insertion. One way to minimize pain and discomfort is to orally administer drugs to the patient. To date the most commonly prescribed oral drugs are opioid analgesia (e.g. Vicodin® and Percocet®), which are controlled substances and have the potential for abuse by patients. Another way to address pain and discomfort is to release a therapeutic agent selected from anti-inflammatory agents, analgesic agents, anesthetic agents, antispasmodic agents, or a combination thereof from the urological device. See U.S. Pat. App. Pub. No. 2003/0224033 to Li et al.

Kidney stones are another common cause of pain within the urinary tract. Kidney stones may contain various combinations of chemicals. For example, the most common type of stone contains calcium in combination with either oxalate or phosphate. These chemicals are part of a person's normal diet and make up important parts of the body, such as bone and muscle. A less common type of stone is caused by infection in the urinary tract. This type of stone is called a struvite or infection stone. Much less common are uric acid and cystine stones. Urolithiasis is the medical term used to describe stones occurring in the urinary tract. Another frequently used term is nephrolithiasis. A ureteral stone (or ureterolithiasis) is a kidney stone found in the ureter. As defined herein, the term “kidney stone” or “stone” encompassses any stone of the urinary tract of any size and of any composition.

Usually, the first symptom of a kidney stone is extreme pain. The pain often begins suddenly when a stone moves in the urinary tract (e.g., in the ureter), causing irritation or blockage, and this pain may continue as the muscles in the urinary tract wall (e.g., the ureteral wall) try to squeeze the stone further down the tract. Renal colic is the name given to the condition that arises from kidney stones and symptoms include pain, frequently severe, often associated with nausea and vomiting.

Extracorporeal shock wave lithotripsy is a common procedure for the treatment of kidney stones in which shock waves that are created outside the body travel are directed into the body where they strike stones. In other common procedures, a shock wave generating device is advanced to the stones through the urinary tract. In either case, shock waves break up the stones into smaller fragments, which may be easily passed through the urinary tract in the urine. In some instances, however, the shattered stone fragments cause discomfort as they pass through the urinary tract, in which case the physician may insert a urological stent into the subject to promote passage of the fragments. Even with such procedures, however, the stone may be difficult to pass, requiring more invasive procedures, for example, percutaneous nephrolithomoty procedures, among others.

### SUMMARY OF THE INVENTION

According to an aspect of the present invention, urological medical devices are provided, which contain one or more urologically beneficial agents selected from alpha-adrenergic blockers, calcium channel blockers, and combinations thereof, among others. The urological devices are adapted for implantation or insertion into a subject's urinary tract (e.g., occupying one or more of the urethra, bladder, ureter and kidney), whereupon at least a portion of the urologically beneficial agent is released. Such agents are urologically beneficial, for example, in that they may relieve pain and/or discomfort associated with the medical device and/or act as stone expulsion agents (i.e., they facilitate stone passage), among other benefits.

According to another aspect of the present invention, a method of treating kidney stones is provided which comprises: (a) diagnosing the presence of kidney stones within a subject and (b) implanting or inserting a urological medical device into the subject which contains at least one urologically beneficial agent that acts a stone expulsion agent. The medical device is adapted to release the at least one urologically beneficial agent in vivo in an amount effective to promote kidney stone expulsion.

Still further enumerated aspects of the invention are enumerated in the following paragraphs:

Aspect 1. A urological medical device comprising a urologically beneficial agent selected from alpha-adrenergic blockers, calcium channel blockers and combinations thereof, said urological medical device being adapted for implantation or insertion into a subject's body whereupon at least a portion of said urologically beneficial agent is released in vivo.

Aspect 2. The urological medical device of Aspect 1, wherein said urological medical device is an elongated solid device.

Aspect 3. The urological medical device of Aspect 1, wherein said urological medical device is an elongated hollow device.

Aspect 4. The urological medical device of Aspect 1, wherein said urological medical device is adapted to be introduced to the subject through a channel of another medical device.

Aspect 5. The urological medical device of Aspect 1, wherein said urological medical device is adapted to be introduced to the subject over a guide wire.

Aspect 6. The urological medical device of Aspect 1, wherein said urological medical device is adapted to take on a coiled configuration within the subject.

Aspect 7. The urological medical device of Aspect 1, wherein said urological medical device is selected from urological stents, stone removal devices, and catheters.

Aspect 8. The urological medical device of Aspect 1, comprising a plurality of differing urologically beneficial agents.

Aspect 9. The urological medical device of Aspect 1, wherein said urologically beneficial agent is released in vivo in an amount effective to promote stone expulsion.

Aspect 10. The urological medical device of Aspect 1, wherein said urologically beneficial agent is released in vivo in an amount effective to reduce pain or discomfort associated with said device.

Aspect 11. The urological medical device of Aspect 1, wherein said device is a ureteral stent and wherein said urologically beneficial agent is released in vivo in an amount effective to promote ureteral smooth muscle relaxation.

Aspect 12. The urological medical device of Aspect 1, wherein said urologically beneficial agent is a calcium channel blocker.

Aspect 13. The urological medical device of Aspect 12, wherein said calcium channel blocker is selected from benzothiazepines, dihydropyridines, arylalkylamines, piperazines, and combinations thereof.

Aspect 14. The urological medical device of Aspect 1, wherein said calcium channel blocker is selected from diltiazem, nicardipine, nifedipine, nimodipine, bepridil, verapamil, mibefradil, pharmaceutically effective salts and esters thereof, and combinations thereof.

Aspect 15. The urological medical device of Aspect 1, wherein said urologically beneficial agent is an alpha-adrenergic blocker.

Aspect 16. The urological medical device of Aspect 1, wherein said urologically beneficial agent is an alpha-1-adrenergic blocker.

Aspect 17. The urological medical device of Aspect 15, wherein said alpha-adrenergic blocker is selected from alfuzosin, doxazosin, prazosin, tamsulosin, terazosin, pharmaceutically effective salts and esters thereof, and combinations thereof.

Aspect 18. The urological medical device of Aspect 1, comprising a supplemental agent selected from corticosteroids, narcotic analgesics, non-narcotic analgesics, local anesthetic agents, antibiotics, and combinations thereof, whereupon at least a portion of said supplemental agent is released in vivo.

Aspect 19. The urological medical device of Aspect 1, comprising an imaging contrast agent.

Aspect 20. The urological medical device of Aspect 1, wherein said medical device comprises a polymeric carrier region that comprises said urologically beneficial agent.

Aspect 21. The urological medical device of Aspect 20, wherein said polymeric carrier region corresponds to a urological medical device body.

Aspect 22. The urological medical device of Aspect 20, wherein said polymeric carrier region is in the form of a layer that at least partially covers an underlying urological medical device body.

Aspect 23. The urological medical device of Aspect 20, wherein said polymeric carrier region comprises a supplemental agent selected from corticosteroids, narcotic analgesics, non-narcotic analgesics, local anesthetic agents, antibiotics, imaging contrast agents, and combinations thereof.

Aspect 24. The urological medical device of Aspect 20, wherein said polymeric carrier region comprises a hydrogel.

Aspect 25. The urological medical device of Aspect 20, wherein said polymeric carrier region comprises a biodegradable polymer.

Aspect 26. The urological medical device of Aspect 20, wherein said polymeric carrier region comprises a polymer selected from polycarbonates, silicone homopolymers and copolymers, polyurethanes, poly(ether-b-amides), and alkene homopolymers and copolymers.

Aspect 27. The urological medical device of Aspect 20, wherein said polymeric carrier region comprises an alkene copolymer selected from ethylene-vinyl acetate copolymers, ethylene-methacrylic acid copolymers, ethylene-acrylic acid copolymers, and styrene-isobutylene copolymers.

37 Aspect 28. A method of treating kidney stones comprising: (a) identifying a subject one or more kidney stones, and (b) implanting or inserting a urological medical device into the subject which comprises a urologically beneficial agent, wherein the medical device is adapted to release the urologically beneficial agent in vivo in an amount effective to promote kidney stone expulsion.

Aspect 29. The method of Aspect 28, wherein the urological medical device is a stent.

Aspect 30. The method of Aspect 28, comprising a plurality of differing urologically beneficial agents.

Aspect 31. The method of Aspect 28, wherein the urologically beneficial agent is muscle relaxant.

Aspect 32. The method of Aspect 31, wherein said urologically beneficial agent is released in vivo in an amount and at a location effective to promote ureteral smooth muscle relaxation.

Aspect 33. The method of Aspect 28, wherein said urologically beneficial agent is a calcium channel blocker.

Aspect 34. The method of Aspect 33, wherein said calcium channel blocker is selected from benzothiazepines, dihydropyridines, arylalkylamines, piperazines, and combinations thereof.

Aspect 35. The method of Aspect 33, wherein said calcium channel blocker is selected from diltiazem, nicardipine, nifedipine, nimodipine, bepridil, verapamil, mibefradil, pharmaceutically effective salts and esters thereof, and combinations thereof.

Aspect 36. The method of Aspect 28, wherein said urologically beneficial agent is an alpha-adrenergic blocker.

Aspect 37. The method of Aspect 28, wherein said urologically beneficial agent is an alpha-1-adrenergic blocker.

Aspect 38. The method of Aspect 36, wherein said alpha-adrenergic blocker is selected from alfuzosin, doxazosin, prazosin, tamsulosin, terazosin, pharmaceutically effective salts and esters thereof, and combinations thereof.

Aspect 39. The method of Aspect 28, wherein said urologically beneficial agent is a beta-adrenergic agonist.

Aspect 40. The method of Aspect 39, wherein said beta-adrenergic agonist is selected from ritodrine, terbutaline, pharmaceutically acceptable salts and esters thereof, and combinations thereof.

Aspect 41. The method of Aspect 28, wherein said urologically beneficial agent is a bronchodilator.

Aspect 42. The method of Aspect 41, wherein said bronchodilator is selected from albuterol and pharmaceutically acceptable salts and esters thereof.

Aspect 43. The method of Aspect 28, wherein said urologically beneficial agent is a cathartic agent.

Aspect 44. The method of Aspect 43, wherein said cathartic agent is a magnesium salt.

Aspect 45. The method of Aspect 28, wherein said urologically beneficial agent is a nitric oxide donor.

Aspect 46. The method of Aspect 45, wherein said nitric oxide donor is nitrogycerin.

Aspect 47. The method of Aspect 28, wherein said urologically beneficial agent is a prostaglandin or a prostaglandin analog.

Aspect 48. The method of Aspect 28, wherein said medical device comprises a polymeric carrier region that comprises said urologically beneficial agent.

Aspect 49. The method of Aspect 28, wherein the medical device is further adapted to release in vivo a supplemental agent selected from corticosteroids, narcotic analgesics, non-narcotic analgesics, local anesthetic agents, antibiotics, and combinations thereof.

Advantages of the present invention are that medical devices may be provided which, among other therapeutic benefits, (a) relieve pain and/or discomfort associated with the medical device, (b) facilitate the passage of kidney stones, or (c) both.

Another advantage of the present invention is that urologically beneficial agents may be applied locally, thereby avoiding the need for systemic drug administration, which typically requires higher quantities of drug to be efficacious. In this regard, virtually all therapeutic agents have side effects.

These and other aspects, embodiments and advantages of the present invention will become immediately apparent to those of ordinary skill in the art upon review of the Detailed Description and Claims to follow.

### BRIEF DESCRIPTION OF THE DRAWING

FIG. 1 is a side view of a ureteral stent, in accordance with an embodiment of the invention.

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention.

In one aspect, the present invention provides implantable or insertable urological medical devices, which are adapted to release one or more urologically beneficial agents in pharmaceutically effective amounts. For example, such agents may be provided in amounts effective to achieve the following benefits, among others: (a) the relief of pain and/or discomfort associated with the medical device and/or (b) the facilitation of kidney stone expulsion. Preferred subjects (also referred to as “patients”) are vertebrate subjects, more preferably mammalian subjects and more preferably human subjects.

As used herein, a “urologically beneficial agent” is an agent that is approved or capable of being approved by the United States Food and Drug Administration or Department of Agriculture as sufficiently safe and effective for use in humans or animals when released from an implantable or insertable urological medical device.

Urological medical devices for use in conjunction with the present invention include any device which is suitable for placement in the urinary tract of a subject, including the kidneys (e.g., in the renal calyx, renal pelvis, etc.), ureters, bladder and urethra. These include various elongated devices including elongated devices having any of a variety of solid and hollow cross-sections including circular (e.g., tubular and rod-shaped devices), oval, triangular, and rectangular (e.g., ribbon-shaped devices), among many other regular and irregular cross sections. Specific examples include urological stents, for example, urethral and ureteral stents, urological catheters (e.g., drainage catheters, guide catheters, etc.), guidewires, urological scopes (e.g., cytoscopes, ureteroscopes, nephroscopes, etc.), tissue engineering scaffolds, grafts, patches, synthetic meshes, paving systems, and injectable implants, among others.

In some embodiments, devices are provided which are adapted to be advanced over a guide wire or advanced through a channel, for example, one associated with a guide catheter or scope.

In some embodiments, devices may be employed that take on a particular beneficial shape in vivo, for example, immediately upon removal of a guide wire or emergence from a channel (e.g., due to elastic rebound of the material) or upon application of an external stimulus such as heat or light (e.g., where a shape memory material such as a shape memory polymer is employed). For example, the device may take on a non-linear form such as a coiled configuration. Such constructions allow the medical device to be held in place in the urinary tract, for example, by forming a coil or other retention, element in the kidney (e.g., in the renal calyx and/or renal pelvis), the bladder, or both.

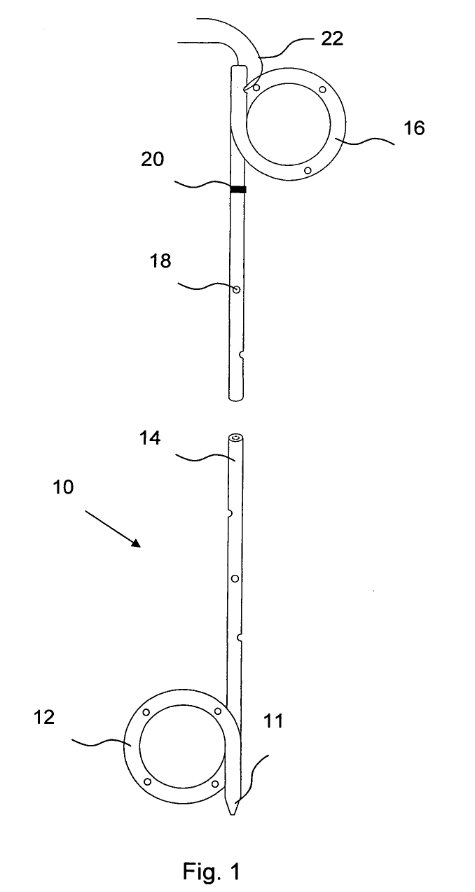

In this regard, ureteral stents are commonly provided with two coils or “pigtails” to keep them properly positioned, with one forming in the bladder and the other forming in the kidney. A schematic diagram of such a stent 10 is illustrated in FIG. 1. The stent 10 is a tubular polymeric extrusion containing a renal pigtail 12, a shaft 14 and a bladder pigtail 16. The stent 10 shown is further provided with the following: (a) a tapered tip 11, to aid insertion, (b) multiple side ports 18 (one numbered), which are arranged in a spiral pattern down the length of the body to promote drainage, (c) graduation marks 20 (one illustrated), which are used for visualization by the physician to know when the appropriate length of stent has been inserted into the ureter, and (d) a Nylon suture 22, which aids in positioning and withdrawal of the stent, as is known in that art. During placement, such ureteral stents 10 are typically placed over a urology guide wire, through a cystoscope and advanced into position with a positioner. Once the proximal end of the stent is advanced into the kidney/renal calyx, the guide wire is removed, allowing pigtails 12, 16 to form in the kidney and bladder.

In accordance with the present invention, the stent 10 also contains one or more urologically beneficial agents.

In some embodiments, urologically beneficial agents for use in the invention have muscle relaxant activity (e.g., they have musculotropic relaxant properties, smooth muscle relaxant properties, etc.).

Urologically beneficial agents for use in the invention may be selected, for example, from suitable members of the following, among others: alpha-adrenergic blockers, calcium channel blockers, beta-adrenergic agonists, bronchodilators, nitric oxide donors, nitric oxide releasing compounds, prostaglandins, cathartic agents, and combinations thereof.

Examples of alpha-adrenergic blockers for use in the present invention may be selected from suitable members of the following: alfuzosin, amosulalol, arotinilol, dapiprazole, doxazosin, ergoloid mesylates, fenspiride, idazoxan, indoramin, labetalol, manotepil, naftopidil, nicergoline, prazosin, tamsulosin, terazosin, tolazoline, trimazosin, and yohimbine, among others, as well as combinations and pharmaceutically acceptable salts, esters and other derivatives of the same. Of these, tamsulosin, alfuzosin, doxazosin, prazosin, tamsulosin and terazosin are alpha-1-adrenergic blockers, of which tamsulosin and alfuzosin are selective alpha-1-adrenergic blockers.

Examples of calcium channel blockers for use in the present invention may be selected from suitable members of the following: arylalkylamines (including phenylalkylamines) such as verapamil, gallopamil, bepridil, clentiazen, fendiline, mibefradil, prenylamine, semotiadil, and terodiline; benzothiazepines such as diltiazem; dihydropyridine derivatives (including 1,4-dihydropyridine derivatives) such as amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine and nitrendipine; piperazine derivatives such as cinnarizine, dotarizine, flunarizine, lidoflazine and lomerizine; other calcium channel blockers such as bencyclane, etafenone, fantofarone, monatepil and perhexiline; among other calcium channel blockers, as well as combinations and pharmaceutically acceptable salts, esters and other derivatives of the same.

Examples of beta-adrenergic agonists for use in the present invention may be selected from suitable members of the following: albuterol, bambuterol, bitolterol, carbuterol, clenbuterol, clorprenaline, denopamine, ephedrine, epinephrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, prenalterol, procaterol, protokylol, reproterol, rimiterol, ritodrine, salmerterol, soterenol, terbutaline, treloquinol, tulobuterol and xamoterol, among others, as well as combinations and pharmaceutically acceptable salts, esters and other derivatives of the same.

Examples of bronchodilators for use in the present invention may be selected from suitable members of the following: (a) ephedrine derivatives such as albuterol, bambuterol, bitolterol, carbuterol, clenbuterol, clorprenaline, dioxethedrine, ephedrine, epinephrine, eprozinol, etafedrine, ethylnorepinephrine, fenoterol, formoterol, hexoprenaline, isoetharine, isoproterenol, mabuterol, metaproterenol, n-methylephedrine, pirbuterol, procaterol, protokylol, reproterol, rimiterol, salmeterol, soterenol, terbutaline and tulobuterol, (b) quaternaly ammonium compounds such as bevonium methyl sulfate, flutropium bromide, ipratropium bromide, oxitropium bromide and tiotropium bromide, (c) xanthine derivatives such as acefylline, acefylline piperazine, ambuphylline, aminophylline, bamifylline, choline theophyllinate, doxofylline, dyphylline, etamiphyllin, etofylline, guaithylline, proxyphylline, theobromine, 1-theobromineacetic acid and theophylline, and (d) other bronchodilators such as fenspiride, medibazine, methoxyphenanime and tretoquinol, among others, as well as combinations and pharmaceutically acceptable salts, esters and other derivatives of the forgoing.

Examples of nitric oxide donors/releasing molecules for use in the present invention may be selected from suitable members of the following: iorganic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, among others, as well as pharmaceutically acceptable salts, esters and other derivatives of the same, and combinations of the foregoing.

Examples of prostaglandins and analogs thereof for use in the present invention may be selected from suitable members of the following: prostaglandins such as PGE1 and PGI2 and prostacyclin analogs such as ciprostene, epoprostenol, carbacyclin, iloprost and beraprost, among others, as well as pharmaceutically acceptable salts, esters and other derivatives of the same, and combinations of the foregoing.

Examples of cathartic agents for use in the present invention may be selected from suitable magnesium salts such as magnesium sulfate, among others.

In addition to one or more urologically beneficial agents, the urological medical devices of the invention may also contain one or more optional supplemental agents (some of which may also have urologically beneficial properties).

Such optional supplemental agents may, include, for example, supplemental therapeutic agents such as corticosteroids, narcotic and non-narcotic analgesics, local anesthetic agents, antibiotics and combinations thereof, among others. Such supplemental therapeutic agents may also be administered independently of urological devices of the invention, for example, by systemic administration or other local modes of administration.

Examples of corticosteroids for use in the present invention may be selected from suitable members of the following: betamethasone, cortisone, dexamethasone, deflazacort, hydrocortisone, methylprednisolone, prednisolone, prednisone and triamcinolone, among others, as well as combinations and pharmaceutically acceptable salts, esters and other derivatives of the same.

Examples of narcotic analgesic agents for use in the present invention may be selected from suitable members of the following: codeine, morphine, fentanyl, meperidine, propoxyphene, levorphanol, oxycodone, oxymorphone, hydromorphone, pentazocine, and methadone, among others, as well as combinations and pharmaceutically acceptable salts, esters and other derivatives of the same.

Examples of non-narcotic analgesic agents for use in the present invention may be selected from suitable members of the following: analgesic agents such as acetamiinophen, and non-steroidal anti-inflammatory drugs such as aspirin, diflunisal, salsalate, ibuprofen, ketoprofen, naproxen indomethacin, celecoxib, valdecoxib, diclofenac, etodolac, fenoprofen, flurbiprofen, ketorolac, meclofenamate, meloxicam, nabumetone, naproxen, oxaprozin, piroxicam, sulindac, tolmetin, and valdecoxib, among others, as well as combinations and pharmaceutically acceptable salts, esters and other derivatives of the same.

Examples of local anesthetic agents for use in the present invention may be selected from suitable members of the following: benzocaine, cocaine, lidocaine, mepivacaine, and novacaine, among others, as well as combinations and pharmaceutically acceptable salts, esters and other derivatives of the same.

Examples of antibacterial agents for use in the present invention may be selected from suitable members of the following: the penicillins (e.g., penicillin G, methicillin, oxacillin, ampicillin, amoxicillin, ticarcillin, etc.), the cephalosporins (e.g., cephalothin, cefazolin, cefoxitin, cefotaxime, cefaclor, cefoperazone, cefixime, ceftriaxone, cefuroxime, etc.), the carbapenems (e.g., imipenem, metropenem, etc.), the monobactems (e.g., aztreonem, etc.), the carbacephems (e.g., loracarbef, etc.), the glycopeptides (e.g., vancomycin, teichoplanin, etc.), bacitracin, polymyxins, colistins, fluoroquinolones (e.g., norfloxacin, lomefloxacin, fleroxacin, ciprofloxacin, enoxacin, trovafloxacin, gatifloxacin, etc.), sulfonamides (e.g., sulfamethoxazole, sulfanilamide, etc.), diaminopyrimidines (e.g., trimethoprim, etc.), rifampin, aminoglycosides (e.g., streptomycin, neomycin, netilmicin, tobramycin, gentamicin, amikacin, etc.), tetracyclines (e.g., tetracycline, doxycycline, demeclocycline, minocycline, etc.), spectinomycin, macrolides (e.g., erythromycin, azithromycin, clarithromycin, dirithromycin, troleandomycin, etc.), and oxazolidinones (e.g., linezolid, etc.), among others, as well as combinations and pharmaceutically acceptable salts, esters and other derivatives of the same.

Many of the above and other urologically beneficial agents and supplemental therapeutic agents may be found, for example, in The Merck Index, 13^{th} Edition, M.J. O'Neil, Senior Editor, published by Merck Research Laboratories, 2001.

Other examples of supplemental agents include imaging agents.

For example, x-ray based fluoroscopy is a diagnostic imaging technique that allows real-time patient monitoring of motion within a patient. To be fluoroscopically visible, devices and/or compositions are typically rendered more absorptive of x-rays than the surrounding tissue (e.g., radiopaque materials). In various embodiments of the invention, this is accomplished by the use of contrast agents. Examples of contrast agents for use in connection with x-ray fluoroscopy include metals, metal salts and oxides (particularly bismuth salts and oxides), and iodinated compounds, among others. More specific examples of such contrast agents include tungsten, platinum, tantalum, iridium, gold, or other dense metal, barium sulfate, bismuth subcarbonate, bismuth trioxide, bismuth oxychloride, metrizamide, iopamidol, iothalamate sodium, iodomide sodium, and meglumine, among others.

Ultrasound uses high frequency sound waves to create an image of living tissue. A sound signal is sent out, and the reflected ultrasonic energy, or “echoes,” are used to create the image. Ultrasound imaging contrast agents are materials that enhance the image produced by ultrasound equipment. Ultrasonic imaging contrast agents can be, for example, echogenic (i.e., materials that result in an increase in the reflected ultrasonic energy) or echolucent (i.e., materials that result in a decrease in the reflected ultrasonic energy). Suitable ultrasonic imaging contrast agents for use in connection with the present invention include solid particles ranging from about 0.01 to 50 microns in largest dimension (e.g., the diameter, where spherical particles are utilized), more typically about 0.5 to 20 microns. Both inorganic and organic particles can be used. Examples include microparticles/microspheres of calcium carbonate, hydroxyapatite, silica, poly(lactic acid), and poly(glycolic acid), among others. Microbubbles can also be used as ultrasonic imaging contrast agents, as is known in the imaging art.

Magnetic resonance imaging (MRI) produces images by differentiating detectable magnetic species in the portion of the body being imaged. In the case of ¹H MRI, the detectable species are protons (hydrogen nuclei). In order to enhance the differentiation of detectable species in the area of interest from those in the surrounding environment, imaging contrast agents are often employed. These agents alter the magnetic environment of the detectable protons in the area of interest relative to that of protons in the surrounding environment and thereby allow for enhanced contrast and better images of the area of interest. For contrast-enhanced MRI, it is desirable that the contrast agent have a large magnetic moment, with a relatively long electronic relaxation time. Based upon these criteria, contrast agents such as Gd(III), Mn(II) and Fe(III) have been employed. Gaclolinium(III) has the largest magnetic moment among these three and is, therefore, a widely-used paramagnetic species to enhance contrast in MRI. Chelates of paramagnetic ions such as Gd-DTPA (gadolinium ion chelated with the ligand diethylenetriaminepentaacetic acid) have been employed as MRI contrast agents. Chelation of the gadolinium or other paramagnetic ion is believed to reduce the toxicity of the paramagnetic metal by rendering it more biocompatible, and can assist in localizing the distribution of the contrast agent to the area of interest. Further information can be found, for example, in U.S. Patent Application No. 2003/0100830 entitled “Implantable or insertable medical devices visible under magnetic resonance imaging,” the disclosure of which is incorporated herein by reference, to the extent that it does not conflict with the present application.

In certain embodiments of the invention, one or more agents (e.g., urologically beneficial agents, optional supplemental agents such as supplemental therapeutic agents and supplemental imaging agents, etc.) are disposed within a polymeric carrier region. As used herein a polymeric carrier region is one that contains one or more polymers and one or more agents, which agent may or may not be released from the polymeric carrier region in vivo. The polymeric carrier region may correspond, for example, to an entire urological medical device or to a portion of a urological medical device. For instance, the polymeric carrier region may be in the form of a medical device body (e.g., a stent body), in the form of a urological medical device component, in the form of one or more fibers which are incorporated into a urological medical device, or in the form of one or more polymeric layers formed over all or only a portion of an underlying substrate (e.g., urological medical device body), among many other possibilities. Layers can be provided over an underlying substrate at a variety of locations and in a variety of shapes (e.g., in the form of a series of rectangles, stripes, or any other continuous or non-continuous pattern). As used herein a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein.

By “polymeric region” is meant a region (e.g., corresponding to a coating layer, a device component, an entire device, etc.) that contains one or more types of polymers. By “carrier region” is meant a region that contains one or more agents, for example, selected from urologically beneficial agents and optional supplemental agents such as those described above, among others. By “polymeric carrier region” is meant a region that contains one or more polymers and one or more agents.

As noted above, a “polymeric” region is one that contains polymers, for example, 50 wt % or lower to 75 wt % to 90 wt % to 95 wt % to 97.5 wt % to 99 wt % polymers, or more.

As used herein, “polymers” are molecules containing multiple copies (e.g., from 2 to 5 to 10 to 25 to 50 to 100 to 250 to 500 to 1000 or more copies) of one or more constitutional units, commonly referred to as monomers.

Polymers may take on a number of configurations, which may be selected, for example, from cyclic, linear, branched and networked (e.g., crosslinked) configurations. Branched configurations include star-shaped configurations (e.g., configurations in which three or more chains emanate from a single branch point, for instance an initiator molecule or a linking molecule), comb configurations (e.g., configurations having a main chain and a plurality of side chains), dendritic configurations (e.g., arborescent and hyperbranched polymers), and so forth.

As used herein, “homopolymers” are polymers that contain multiple copies of a single constitutional unit. “Copolymers” are polymers that contain multiple copies of at least two dissimilar constitutional units, examples of which include random, statistical, gradient, periodic (e.g., alternating) and block copolymers. As used herein, “block copolymers” are copolymers that contain two or more polymer blocks that differ in composition, for instance, because a constitutional unit (i.e., monomer) is found in one polymer block that is not found in another polymer block. As used herein, a “polymer block” is a grouping of constitutional units (e.g., 2 to 5 to 10 to 25 to 50 to 100 to 250 to 500 to 1000 or more units). Blocks can be branched or unbranched, and they may be networked (e.g., by crosslinking). Blocks can contain a single type of constitutional unit (also referred to herein as “homopolymeric blocks”) or multiple types of constitutional units (also referred to herein as “copolymeric blocks”) which may be provided, for example, in a random, statistical, gradient, or periodic (e.g., alternating) distribution.

Polymers for use in the present invention may be selected, for example, from various thermoplastic, elastomeric, and thermoplastic-elastomeric polymers.

Polymers for use in the present invention may be selected, for example, from polycarbonates, silicone polymers, polyurethanes, poly(ether-block-amides), and alkene polymers.

Polycarbonates are derived from the reaction of carbonic acid derivatives with aromatic, aliphatic, or mixed diols. They may be produced, for example, by the reaction of phosgene with a diol in the presence of an appropriate hydrogen chloride receptor or by a melt transesterification reaction between a diol and a carbonate ester. Polycarbonates can be made from a wide variety of starting materials. For example, a common polycarbonate, bisphenol A polycarbonate, is a polycarbonate made by reacting bisphenol A with phosgene by condensation. For further information, see, e.g., U.S. Pat. No. 5,580,924 and the references cited therein.

Silicone polymers (also referred to as polysiloxanes) are polymers comprising one or more types of siloxane units,

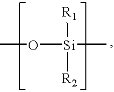

where R₁ and R₂ can be the same or different and may be selected from linear, branched and cyclic alkyl groups, aromatic groups and alky-aromatic groups, for example, having from 1 to 10 carbon atoms and having 5 or more, typically 10 to 25 to 50 to 100 to 250 to 500 to 1000 or more siloxane units. Examples include polydimethylsiloxane, polydiethylsiloxane, polymethylethylsiloxane, polymethylphenylsiloxane, and polydiphenylsiloxane, among many others.

In general, polyurethanes are a family of polymers that are synthesized from polyfunctional isocyanates (e.g., diisocyanates, including both aliphatic and aromatic diisocyanates) and polyols (also, referred to as macroglycols, e.g., macrodiols). Commonly employed macroglycols include polyester glycols, polyether glycols and polycarbonate glycols. Typically, aliphatic or aromatic diols are also employed as chain extenders, for example, to impart the useful physical properties described above. Examples of diol chain extenders include butane diol, pentane diol, hexane diol, heptane diol, benzene dimethanol, hydraquinone diethanol and ethylene glycol. Polyurethanes are commonly classified based on the type of macroglycol employed, with those containing polyester glycols being referred to as polyester polyurethanes, those containing polyether glycols being referred to as polyether polyurethanes, and those containing polycarbonate glycols being referred to as polycarbonate polyurethanes. Polyurethanes are also commonly designated aromatic or aliphatic on the basis of the chemical nature of the diisocyanate component in their formulation. For example, U.S. Patent App. No. 2004/0131863 to Belliveau et al. describes aliphatic polycarbonate polyurethanes which are the reaction products of (a) a hydroxyl terminated polycarbonate, (b) an aliphatic diisocyanate and (c) a lower aliphatic chain extender. Hydroxyl terminated polycarbonate polyol may be prepared by reacting a glycol with a carbonate, as disclosed in U.S. Pat. No. 4,131,731. Suitable aliphatic diisocyanates include hexamethylene diisocyanate (HDI), isophorone diisocyanate (IPDI), trimethyl hexamethylene diisocyanate (TMHDI), dicyclohexyl methane diisocyanate (HMDI), and dimer acid diisocyanate (DDI), with HMDI said to be preferred. Suitable chain extenders include lower aliphatic glycols having from about 2 to about 10 carbon atoms, such as, for instance ethylene glycol, diethylene glycol, propylene glycol, dipropylene glycol, 1,4-butanediol, 1,6-hexanediol, 1,3-butanediol, 1,5-pentanediol, 1,4-cyclohexanedimethanol hydroquinone di(hydroxyethyl)ether, neopentyglycol, and the like, with 1,4-butanediol said to be preferred.

Another group of polymers are block copolymers comprising polyether blocks (i.e., polymer blocks containing multiple C—O—C linkages) and polyamide blocks (i.e., polymer blocks containing multiple —NH—CO— linkages), sometimes referred to as poly(ether-b-amides) or polyether-block-amides. A few specific examples of polyether blocks include homopolymeric and copolymeric blocks of the formulas (a-[R₁—O_{]}ₙ— or (b)-[R₁—O—R₂—O]ₙ—, where R₁ and R₂ can be the same or different and may be selected from linear, branched and cyclic alkyl groups, aromatic groups and alky-aromatic groups, for example, having from 1 to 10 carbon atoms (more typically linear or branched alkyl groups having from 1 to 6 carbons) and where n is an integer of 5 or more, typically 10 to 25 to 50 to 100 to 250 to 500 to 1000 or more. Polyethers may be formed, for example, from ring opening addition polymerization of cyclic ethers, such as ethylene oxide, where R₃═R₂=dimethylene (i.e., [—(CH₂)₂—]ₙ), which is commonly referred to as polyethylene glycol or as polyethylene oxide), trimnethylene oxide, where R₁=R₂=trimethylene (i.e., [ (CH₂)₃—O—]ₙ), propylene oxide, where R₁=R₂=methyl substituted dimethylene (i.e., [—CH₂CH₂(CH₃)—O—]ₙ, referred to as polypropylene glycol or polypropylene oxide), and tetrahydrofuran, where R₁═R₂=tetramethylene (i.e., —[(CH₂)₄—O]—ₙ, which is referred to as polytetramethylene glycol, polytetramethylene oxide (PTMO), or terathane). Examples of polyamide blocks, which may be provided, for example, as homopolymeric or copolymeric blocks, include polyamides of the formula —[R₃—NH—CO]ₘ— or —[NH—R₃—NH—CO—R₄—CO]ₘ—, where R₃ and R₄ can be the same or different and may be selected from linear, branched and cyclic alkyl groups, aromatic groups and alky-aromatic groups, for example, of 1 to 20 carbon atoms (more typically linear or branched alkyl groups having from 1 to 15 carbons, such as methyl, ethyl, propyl, isopropyl, and so forth) and where m is an integer of 5 or more, typically 10 to 25 to 50 to 100 to 250 to 500 to 1000 or more. Specific examples include nylons, such as nylon 6, nylon 4/6, nylon 6/6, nylon 6/10, nylon 6/12, nylon 11 and nylon 12. A specific example of a polyether-polyamide block copolymer is poly(tetramethylene oxide)-b-polyamide-12 block copolymer, available from Elf Atochem as PEBAX.

Further polymers include polyalkene homopolymers and copolymers with themselves and with various other monomers including those selected from vinyl aromatic monomers such as styrene, acrylic acid, methacrylic acid, and vinyl acetate. Examples of alkene monomers include ethylene, propylene, isobutylene, 1-butene, 1-pentene, 4-methyl-1-pentene, dienes such as 1,3-butadiene, 2-methyl-1,3-butadiene (isoprene), 2,3-dimethyl-1,3-butadiene, 2-ethyl-1,3-butadiene, 1,3-pentadiene, 2-methyl-1,3-pentadiene, 4-butyl-1,3-pentadiene, 2,3-dibutyl-1,3-pentadiene, 2-ethyl-1,3-pentadiene, 1,3-hexadiene, 1,3-octadiene, and 3-butyl-1,3-octadiene, among others. Specific examples of alkene copolymers include, poly(ethylene-co-vinyl acetate) (EVA), poly(ethylene-co-methacrylic acid), poly(ethylene-co-acrylic acid), and poly(isobutylene-co-styrene), among many others. Among EVA copolymers are included random and other copolymers having a vinyl acetate weight percent ratio of from about 0.5% to 1% to 2% to 5% to 15% to 20% to 30% to 40% or more. In general, the higher the vinyl acetate content, the lower the stiffness and Durometer of the EVA. Thus, the stiffness and durometer may be varied within the device, in certain embodiments. Taking a ureteral stent as an example, a stent may be produced having distinct end regions of different durometer value with a transitional region in between.

Further polymers for use in the present invention may be selected, for example, from suitable members of the following (which polymers are not necessarily exclusive of those described above): polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-alkylene copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends, further copolymers and derivatives of the above, among others.

In certain embodiments, biodegradable polymers are employed in the present invention, which may include for example, polyesters, polyanhydrides, and/or amino acid based polymers, among others. Specific biodegradable polymers may be selected from suitable members of the following (which are not necessarily exclusive of the polymers listed above), among others: (a) polyester homopolymers and copolymers such as polyglycolide, poly-L-lactide, poly-D-lactide, poly-D,L-lactide, poly(beta-hydroxybutyrate), poly-D-gluconate, poly-L-gluconate, poly-D,L-gluconate, poly(epsilon-caprolactone), poly(delta-valerolactone), poly(p-dioxanone), poly(trimethylene carbonate), poly(lactide-co-glycolide) (PLGA), poly(lactide-co-delta-valerolactone), poly(lactide-co-epsilon-caprolactone), poly(lactide-co-beta-malic acid), poly(lactide-co-trimethylene carbonate), poly(glycolide-co-trimethylene carbonate), poly(beta-hydroxybutyrate-co-beta-hydroxyvalerate), poly[1,3-bis(p-carboxyphenoxy)propane-co-sebacic acid], poly(sebacic acid-co-fumaric acid), and poly(ortho esters) such as those synthesized by copolymerization of various diketene acetals and diols, among others, (b) polyanhydride homopolymers and copolymers such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides]such as poly[1,3-bis(p-carboxyphenoxy)propane anhydride]and poly[1,3-bis(p-carboxyphenoxy)hexane anhydride], among others; and (c) amino-acid-based homopolymers and copolymers including tyrosine-based polyarylates (e.g., copolymers of a diphenol and a diacid linked by ester bonds, with diphenols selected, for instance, from ethyl, butyl, hexyl, octyl and bezyl esters of desaminotyrosyl-tyrosine and diacids selected, for instance, from succinic, glutaric, adipic, suberic and sebacic acid), tyrosine-based polycarbonates (e.g., copolymers formed by the condensation polymerization of phosgene and a diphenol selected, for instance, from ethyl, butyl, hexyl, octyl and bezyl esters of desaminotyrosyl-tyrosine), and leucine and lysine-based polyester-amides; specific examples of tyrosine based polymers include poly(desaminotyrosyl-tyrosine ethyl ester adipate) or poly(DTE adipate), poly(desaminotyrosyl-tyrosine hexyl ester succinate) or poly(DTH succinate), poly(desaminotyrosyl-tyrosine ethyl ester carbonate) or poly(DTE carbonate), poly(desaminotyrosyl-tyrosine butyl ester carbonate) or poly(DTB carbonate), poly(desaminotyrosyl-tyrosine hexyl ester carbonate) or poly(DTH carbonate), and poly(desaminotyrosyl-tyrosine octyl ester carbonate) or poly(DTO carbonate).

In certain embodiments, hydrogel polymers are employed in the present invention. These include, for example, hydrogel polymers disclosed in U.S. Pat. Nos. 6,316,522, 6,261,630, 6,184,266, 6,176,849, 6,096,018, 6,060,534, 5,702,754, 5,693,034 and 5,304,121, the disclosures of which are hereby incorporated by reference, to the extent that they do not conflict with the present application. Specific examples of hydrogel polymers, not necessarily exclusive of the polymers in the prior paragraph, include polyacrylates, poly(acrylic acid), poly(methacrylic acid), polyacrylamides, poly(N-alkylacrylamides), polyalkylene oxides such as poly(ethylene oxide) and poly(propylene oxide), poly(vinyl alcohol), poly(vinyl aromatics), poly(vinylpyrrolidone), poly(ethylene imine), poly(ethylene amine), polyacrylonitrile, poly(vinyl sulfonic acid), polyamides, poly(L-lysine), hydrophilic polyurethanes, maleic anhydride polymers, proteins, collagen, cellulosic polymers, methyl cellulose, carboxymethyl cellulose, dextran, carboxymethyl dextran, modified dextran, alginates, alginic acid, pectinic acid, hyaluronic acid, chitin, pullulan, gelatin, gellan, xanthan, carboxymethyl starch, chondroitin sulfate, guar, starch, and blends, copolymers, and derivatives thereof, among others.

Various methods of crosslinking hydrogel polymers are known and include, for instance, (a) covalent crosslinking, for example, with polyfunctional crosslinking agents that bridge hydrogel polymer chains by reaction with functional groups along the hydrogel polymer chains and/or (b) ionic crosslinking, for example, using polyvalent ions. Other crosslinking methods, such as crosslinking by exposing the hydrogel polymer to light of an appropriate frequency, may also be employed. Thus, hydrogel polymers useful in accordance with the present invention may be ionically crosslinked, covalently crosslinked, tonically and covalently crosslinked, or crosslinked by other methods known in the art. A polyfunctional crosslinking agent may be any compound having at least two functional groups that react with functional groups in the hydrogel polymer. Crosslinking ions that are used to ionically crosslink the hydrogel polymers may be anions or cations, depending on whether the polymer is anionically or cationically crosslinkable. Covalent and ionic crosslinking agents are well known in the hydrogel art.

A wide range of agent loadings (e.g., selected from urologically beneficial agents and optional supplemental agents such as optional therapeutic agents, imaging agents, etc.) may be used in conjunction with the urological medical devices of the present invention, with the effective amount being readily determined by those of ordinary skill in the art. For a polymeric carrier region, typical loadings range, for example, from than 1 wt % or less to 2 wt % to 5 wt % to 10 wt % to 25 wt % to 50 wt % or more.

The release profile of the one or more urologically beneficial agents from the device (as well as the release profile of any optional supplemental agents), will be affected by a number of variables. For example, where a polymeric carrier region is utilized, the release profile will depend upon the particular agent(s) selected, the particular polymer(s) that are selected, and their relative amounts. The release profile will also be affected by the size, number and/or position of the polymeric carrier regions within the device. For example, the release profile may be modified by varying the thickness or surface area of the polymeric carrier region. Moreover, multiple polymeric carrier regions may be employed. For example, multiple polymeric carrier regions having the same or different content (e.g., different polymeric content and/or different agent content) may be positioned laterally with respect to one another. Alternatively, a polymeric layer (e.g., formed from one or more polymers described above, either with or without additional agents) may be positioned over a polymeric carrier region in accordance with the invention, thereby acting as a barrier layer.

In some embodiments, the release profile may be modified by increasing the rate at which the polymeric region absorbs water from the surrounding environment, for example, by employing a rapidly hydrating polymer (e.g., a hydrogel) or a rapidly hydrating polymer block (or by varying the ratio of a rapidly hydrating polymer or polymer block vis-à-vis a slowly hydrating polymer or polymer block, respectively), by the addition of an osmotic agent such as a soluble salt or sugar excipient as an optional supplemental agent, and so forth.

Numerous techniques are available for forming polymeric carrier regions in accordance with the present invention.

For example, where the polymeric carrier region is formed from one or more polymers having thermoplastic characteristics, a variety of standard thermoplastic processing techniques may be used to form the polymeric carrier region, including injection molding, compression molding, blow molding, spinning, vacuum forming and calendaring, extrusion into sheets, fibers, rods, tubes and other cross-sectional profiles of various lengths, and combinations of these processes. Using these and other thermoplastic processing techniques, entire devices or portions thereof can be fonned.

141 In some embodiments, mixing or compounding the one or more polymers making up the carrier region and one or more agents (e.g., selected from urologically beneficial agents and optional supplemental agents) may be performed using any suitable processing technique known in the art. For example, where thermoplastic materials are employed, a polymer melt may be formed. A common way of doing so is to apply mechanical shear to a mixture of the polymer(s) and the agent(s). After compounding, the material may be processed using, for example, one or more of the thermoplastic techniques described above, among others.

Other processing techniques besides thermoplastic processing techniques may also be used to form the polymeric carrier regions of the present invention, including solvent-based techniques. Using these techniques, a polymeric carrier region can be formed by (a) first providing a solution or dispersion that contains (i) solvent, (ii) polymer(s), (iii) urologically beneficial agent(s), and (iv) any optional supplemental agent(s), and (b) subsequently removing the solvent. The solvent that is ultimately selected will contain one or more solvent species (e.g., water and/or one or more organic solvents), which are generally selected based on their ability to dissolve the polymer(s) that form the polymeric carrier region (and in many embodiments the urologically beneficial agent(s) and any optional supplemental agent(s)), in addition to other factors, including drying rate, surface tension, etc. Preferred solvent-based techniques include, but are not limited to, solvent casting techniques, spin coating techniques, web coating techniques, solvent spraying techniques, dipping techniques, techniques involving coating via mechanical suspension including air suspension, ink jet techniques, electrostatic techniques, and combinations of these processes.

In certain embodiments of the invention, a polymer-containing solution (where solvent-based processing is employed) or a polymer melt (where thermoplastic processing is employed) is applied to a substrate to form a polymeric carrier region, which solution or melt may also contain urologically beneficial agent(s) and/or any optional supplemental agent(s). For example, the substrate can correspond to all or a portion of an implantable or insertable urological medical device body to which a polymeric carrier region is applied. The substrate can also be, for example, a template, such as a mold, from which the polymeric carrier region is removed after solidification. In certain other embodiments, for example, extrusion and co-extrusion techniques, one or more polymeric carrier regions are formed without the aid of a substrate. In a more specific example, an entire stent body may be extruded as a carrier region. In another, a polymeric carrier layer may be co-extruded along with an underlying stent body. In another, a polymeric carrier layer may be provided by spraying or extruding a coating layer onto a pre-existing stent body. In yet another more specific example, a stent body may be cast in a mold.

As seen from the above, where various agents-for example, urologically beneficial agent(s) and/or any optional supplemental agent(s)-are stable under the polymer processing conditions employed, then they can be combined with the polymers and co-processed along with the same to form the polymeric carrier region of interest. Alternatively, the agent or agents of choice can be introduced subsequent to the formation of the polymeric region using techniques such as imbibing (e.g., where the agent or agents of choice are dissolved or dispersed in a solvent and then contacted with the device, for instance, by spraying, dipping, etc.).

In certain embodiments, the polymeric carrier regions may be crosslinked using methods known in the art, for example, to render them water insoluble.

As noted above, at least one polymeric barrier layer may be provided over a carrier region in accordance with an embodiment of the invention. Such barrier layers may be formed, for example, from the polymer listed above, among others. In these embodiments, the polymeric barrier layer may be formed over the carrier region, for example, using one of the solvent based or thermoplastic techniques described above. Alternatively, a previously formed polymeric barrier region may be adhered over a carrier region.

Although various embodiments are specifically illustrated and described herein, it will be appreciated that modifications and variations of the present invention are covered by the above teachings and are within the purview of the appended claims without departing from the spirit and intended scope of the invention.

1. A urological medical device comprising a urologically beneficial agent selected from alpha-adrenergic blockers, calcium channel blockers and combinations thereof, said urological medical device being adapted for implantation or insertion into a subject's body whereupon at least a portion of said urologically beneficial agent is released in vivo.

2. The urological medical device of claim 1, wherein said urological medical device is an elongated solid device.

3. The urological medical device of claim 1, wherein said urological medical device is an elongated hollow device.

4. The urological medical device of claim 1, wherein said urological medical device is adapted to be introduced to the subject through a channel of another medical device.

5. The urological medical device of claim 1, wherein said urological medical device is adapted to be introduced to the subject over a guide wire.

6. The urological medical device of claim 1, wherein said urological medical device is adapted to take on a coiled configuration within the subject.

7. The urological medical device of claim 1, wherein said urological medical device is selected from urological stents, stone removal devices, and catheters.

8. The urological medical device of claim 1, comprising a plurality of differing urologically beneficial agents.

9. The urological medical device of claim 1, wherein said urologically beneficial agent is released in vivo in an amount effective to promote stone expulsion.

10. The urological medical device of claim 1, wherein said urologically beneficial agent is released in vivo in an amount effective to reduce pain or discomfort associated with said device.

11. The urological medical device of claim 1, wherein said device is a ureteral stent and wherein said urologically beneficial agent is released in vivo in an amount effective to promote ureteral smooth muscle relaxation.

12. The urological medical device of claim 1, wherein said urologically beneficial agent is a calcium channel blocker.

13. The urological medical device of claim 12, wherein said calcium channel blocker is selected from benzothiazepines, dihydropyridines, arylalkylamines, piperazines, and combinations thereof.

14. The urological medical device of claim 1, wherein said calcium channel blocker is selected from diltiazem, nicardipine, nifedipine, nimodipine, bepridil, verapamil, mibefradil, pharmaceutically effective salts and esters thereof, and combinations thereof.

15. The urological medical device of claim 1, wherein said urologically beneficial agent is an alpha-adrenergic blocker.

16. The urological medical device of claim 1, wherein said urologically beneficial agent is an alpha-1-adrenergic blocker.

17. The urological medical device of claim 15, wherein said alpha-adrenergic blocker is selected from alfuzosin, doxazosin, prazosin, tamsulosin, terazosin, pharmaceutically effective salts and esters thereof, and combinations thereof.

18. The urological medical device of claim 1, comprising a supplemental agent selected from corticosteroids, narcotic analgesics, non-narcotic analgesics, local anesthetic agents, antibiotics, and combinations thereof, whereupon at least a portion of said supplemental agent is released in vivo.

19. The urological medical device of claim 1, comprising an imaging contrast agent.

20. The urological medical device of claim 1, wherein said medical device comprises a polymeric carrier region that comprises said urologically beneficial agent.

21. The urological medical device of claim 20, wherein said polymeric carrier region corresponds to a urological medical device body.

22. The urological medical device of claim 20, wherein said polymeric carrier region is in the form of a layer that at least partially covers an underlying urological medical device body.

23. The urological medical device of claim 20, wherein said polymeric carrier region comprises a supplemental agent selected from corticosteroids, narcotic analgesics, non-narcotic analgesics, local anesthetic agents, antibiotics, imaging contrast agents, and combinations thereof.

24. The urological medical device of claim 20, wherein said polymeric carrier region comprises a hydrogel.

25. The urological medical device of claim 20, wherein said polymeric carrier region comprises a biodegradable polymer.

26. The urological medical device of claim 20, wherein said polymeric carrier region comprises a polymer selected from polycarbonates, silicone homopolymers and copolymers, polyurethanes, poly(ether-b-amides), and alkene homopolymers and copolymers.

27. The urological medical device of claim 20, wherein said polymeric carrier region comprises an alkene copolymer selected from ethylene-vinyl acetate copolymers, ethylene-methacrylic acid copolymers, ethylene-acrylic acid copolymers, and styrene-isobutylene copolymers.

28. A method of treating kidney stones comprising: (a) identifying a subject with one or more kidney stones, and (b) implanting or inserting a urological medical device into the subject which comprises a urologically beneficial agent, wherein the medical device is adapted to release the urologically beneficial agent in vivo in an amount effective to promote kidney stone expulsion.

29. The method of claim 28, wherein the urological medical device is a stent.

30. The method of claim 28, comprising a plurality of differing urologically beneficial agents.

31. The method of claim 28, wherein the urologically beneficial agent is muscle relaxant.

32. The method of claim 31, wherein said urologically beneficial agent is released in vivo in an amount and at a location effective to promote ureteral smooth muscle relaxation.

33. The method of claim 28, wherein said urologically beneficial agent is a calcium channel blocker.

34. The method of claim 33, wherein said calcium channel blocker is selected from benzotbiazepines, dihydropyridines, arylalkylamines, piperazines, and combinations thereof.

35. The method of claim 33, wherein said calcium channel blocker is selected from diltiazem, nicardipine, nifedipine, nimodipine, bepridil, verapamil, mibefradil, pharmaceutically effective salts and esters thereof, and combinations thereof.

36. The method of claim 28, wherein said urologically beneficial agent is an alpha-adrenergic blocker.

37. The method of claim 28, wherein said urologically beneficial agent is an alpha-1-adrenergic blocker.

38. The method of claim 36, wherein said alpha-adrenergic blocker is selected from alfuzosin, doxazosin, prazosin, tamsulosin, terazosin, pharmaceutically effective salts and esters thereof, and combinations thereof.

39. The method of claim 28, wherein said urologically beneficial agent is a beta-adrenergic agonist.

40. The method of claim 39, wherein said beta-adrenergic agonist is selected from ritodrine, terbutaline, pharmaceutically acceptable salts and esters thereof, and combinations thereof.

41. The method of claim 28, wherein said urologically beneficial agent is a bronchodilator.

42. The method of claim 41, wherein said bronchodilator is selected from albuterol and pharmaceutically acceptable salts and esters thereof.

43. The method of claim 28, wherein said urologically beneficial agent is a cathartic agent.

44. The method of claim 43, wherein said cathartic agent is a magnesium salt.

45. The method of claim 28, wherein said urologically beneficial agent is a nitric oxide donor.

46. The method of claim 45, wherein said nitric oxide donor is nitrogycerin.

47. The method of claim 28, wherein said urologically beneficial agent is a prostaglandin or a prostaglandin analog.

48. The method of claim 28, wherein said medical device comprises a polymeric carrier region that comprises said urologically beneficial agent.

49. The method of claim 28, wherein the medical device is further adapted to release in vivo a supplemental agent selected from corticosteroids, narcotic analgesics, non-narcotic analgesics, local anesthetic agents, antibiotics, and combinations thereof.
